(19) United States
(12) Patent Application Publication
(10) Pub. No.: US 2014/0373193 A1
(43) Pub. Date: Dec. 18, 2014

(54) Use of OsPP18 Gene in Controlling Rice Drought Resistance

(71) Applicant: HUAZHONG AGRICULTURAL UNIVERSITY, Wuhan (CN)

(72) Inventors: Lizhong Xiong, Wuhan (CN); Jun You, Wuhan (CN)

(21) Appl. No.: 14/351,804

(22) PCT Filed: Oct. 19, 2012
(86) PCT No.: PCT/CN2012/083242

§ 371 Date: Apr. 14, 2014

(30) Foreign Application Priority Data
Oct. 21, 2011 (CN) 2011 10321661.0

(51) Int. Cl. C12N 15/82

(52) U.S. Cl. CPC C12N 15/8273
USPC 800/278; 435/320.1; 435/419; 435/412; 435/415; 435/416; 800/298; 800/320.1; 800/312; 800/314; 800/320.2; 800/320; 800/320.3; 800/322

(57) ABSTRACT

The use of OsPP18 gene in the genetic improvement of drought resistance of a plant is provided. A plant or plant cell comprising a recombinant DNA molecule comprising a polynucleotide encoding OsPP18, a method for producing the plant, a DNA construct comprising a polynucleotide encoding OsPP18 are also provided.

### TECHNICAL FIELD

The present invention relates to the field of plant genetic engineering, in particular relates to the isolation, cloning, and function verification of rice OsPP18 gene which confers enhanced drought resistance, and to the use of the gene in the genetic improvement of rice drought resistance. In the present invention, by screening the candidate genes, OsPP18 gene which controls drought resistance in rice was cloned. Co-segregation test indicates that OsPP18 mutant is closely correlated to the drought-sensitive phenotype. Overexpression of OsPP18 gene improves the drought resistance of transgenic rice, validating the function and use of the gene.

### BACKGROUND ART

Plants are influenced by a number of environmental factors throughout their growth. Drought, cold, and heat can result in massive reduction of crop production, therefore becoming the bottleneck of the agricultural development in many areas. The discovery and development of crops resistant to stresses have thus been one of the major objectives of the agricultural technology researches. To resist or adapt to said adverse factors, the plants can perceive the changes of the extracellular environment and transmit the perception intracellularly through multiple signaling pathways, inducing the expression of several response genes. The expression of these response genes may produce functional proteins that can protect the cells from the adverse impacts of drought, cold, or heat, and may produce osmotic regulation substances as well as transcription factors that can transduce signals and regulate gene expression, therefore acting appropriately in response to the extracellular changes (Xiong et al., Cell signaling during cold, drought and salt stress, Plant Cell. 14 (suppl), S165-S183, 2002). The correct expression of the functional genes responsive to the environmental changes is precisely regulated by the regulatory factors. As one of regulatory factors, transcription factors can initiate the expression of a series of downstream genes under adverse conditions, and enhance stress tolerance in the plants, therefore enabling the plants to resist negative impacts. The abiotic stress signaling in plants involves a variety of transcription factors, such as AP2/EREBP, bZip, HD-ZIP, MYB, MYC, NAC, and Zinc finger transcription factors (Yamaguchi-Shinozaki K, Shinozaki K. Transcriptional regulatory networks in cellular responses and tolerance to dehydration and cold stresses. Annu Rev Plant Biol, 2006, 57: 781-803). Through genetic engineering, some of such transcription factors involved in stress signaling have been successfully used in the genetic development of rice varieties that are resistant to stresses. Overexpression of SNAC1 can increase the seed setting rate by 30% in transgenic rice in the field under drought stress conditions while showing no phenotypic changes or yield penalty under normal conditions. The transgenic rice also shows significantly improved drought resistance and salt tolerance at the vegetative stage (Hu et al., Overexpression a NAM, ATAF, and CUC (NAC) transcription factor enhances drought resistance and salt tolerance in rice. Proc Natl Acad Sci U.S.A., 2006, 103: 12987-12992). The stress responsive transcription factors carry out their functions by regulating the expression of a large number of downstream genes. These downstream genes usually include regulatory proteins that are involved in signal transduction and gene expression, thus forming a secondary regulatory network. Some of these downstream genes might also be used in the genetic development of crops showing resistance to the adverse impacts. Overexpression of HsfA3, a downstream gene of the heat-shock transcription factor DREB2A in Arabidopsis, can augment the heat resistance of the transgenic plants (Yoshida et al., Functional analysis of an Arabidopsis heat-shock transcription factor HsfA3 in the transcirptional cascade downstream of the DREB2A stress-regulatory system. Biochem Biophys Res Commun, 2008, 368: 515-21).

Besides the relevant transcription factors, a number of regulator factors are also involved in the perception and transmission of stress signals so that the plant may respond to the surrounding environment in a precise and timely manner. Reversible phosphorylation of proteins mediated by a protein kinase and a protein phosphatase is an important mechanism that occurs during signal transduction. In phosphorylation, a protein kinase phosphorylates the substrate protein by adding one phosphate group, whereas a protein phosphatase dephosphorylates the substrate protein by removing the phosphate group and therefore carrying out an opposite function. Many enzymes can be activated or deactivated by the addition or removal of the phosphate group. In this way, protein kinases and protein phosphatases play important roles in regulating the enzymes' activities, and further regulate the biological processes participated by such enzymes. Protein phosphatase 2C is a serine/threonine phosphatase. As a neutralizer of protein kinases, protein phosphatase 2C serves a certain function in the signal transduction related to stress response and development (Rodriguez. Protein phosphatase 2C (PP2C) function in higher plants. Plant Mol Biol, 1998, 38: 919-27). The protein phosphatase 2C family in rice comprises 90 members. However, no protein phosphatase 2C that is responsive to abiotic stresses in rice has yet been reported. OsPP18 gene in the present invention is one of the downstream genes of SNAC 1.

Rice is an important food crop and a model plant. At the present day, extreme weather occurs frequently. Therefore, the development of rice with enhanced stress resistance has significant meanings. Although OsPP18 is one of the many downstream genes of the drought resistance gene SNAC1, no report indicates that OsPP18 would be able to enhance the stress resistance in rice.

### SUMMARY OF THE INVENTION

One objective of the present invention relates to the use of OsPP18 gene, a member of the protein phosphatase 2C family, in controlling the improvement of rice stress resistance. The candidate gene was selected among all the genes that were up-regulated in the plants overexpressing SNAC1. Since the candidate gene belongs to the protein phosphatase 2C family, the applicants named it OsPP18. The present invention relates to the isolation and use of a DNA fragment comprising OsPP18 gene, which confers rice the enhanced resistance to drought under drought conditions. The nucleic acid sequence of said DNA fragment that comprises OsPP18 gene is provided in SEQ ID NO:1, with 1162 bp in length; the corresponding amino acid sequence is set forth in SEQ ID NO:1 and has 348 amino acids. The protein sequence of the fragment is set forth in SEQ ID NO: 2. The homologs of SEQ ID NO: 2 are set forth in SEQ ID NOs: 3, 4, and 5.

Therefore, the present invention relates to the following embodiments among others:

Item 1. Use of OsPP18 gene in the genetic improvement of drought resistance of a plant, wherein said gene encodes a protein comprising an amino acid sequence of SEQ ID NO: 2 or an allelic variant or a fragment thereof.

Item 2. The use according to Item 1, wherein said gene has a coding sequence as shown from nt position 38 to nt position 1084 of SEQ ID NO. 1.

Item 3. The use according to Item 1, wherein said gene comprises a nucleotide sequence of SEQ ID NO.1.

Item 4. The use according to any of Items 1-3, wherein said plant is selected from the group consisting of corn, soybean, cotton, canola, rice, barley, oats, wheat, turf grass, alfalfa, sugar beet, sunflower, quinoa and sugar cane.

Item 5. A plant or plant cell comprising a recombinant DNA molecule comprising a polynucleotide encoding a polypeptide, wherein the nucleotide sequence of the polynucleotide is selected from the group consisting of:
a. a nucleotide sequence set forth as SEQ ID NO: 1, or nt position 38 to nt position 1084 of SEQ ID NO: 1;
b. a nucleotide sequence with at least 90% identity to nt position 38 to nt position 1084 of SEQ ID NO: 1;
c. a nucleotide sequence encoding a protein having the amino acid sequence of SEQ ID NO: 2, 3, 4, or 5;
d. a nucleotide sequence encoding an allelic variant or a fragment of SEQ ID NO: 2; and
e. a nucleotide sequence encoding a protein with at least 90% identity to SEQ ID NO: 2;
wherein said plant has improved drought resistance as compared to a control plant.

Item 6. The plant or plant cell of Item 5, wherein said plant is selected from the group consisting of corn, soybean, cotton, canola, rice, barley, oats, wheat, turf grass, alfalfa, sugar beet, sunflower, quinoa and sugar cane.

Item 7. A method for producing a plant comprising:
introducing into a plant cell a recombinant DNA molecule comprising a polynucleotide encoding a polypeptide, wherein the nucleotide sequence of the polynucleotide is selected from the group consisting of
a. a nucleotide sequence set forth as SEQ ID NO: 1, or nt position 38 to nt position 1084 of SEQ ID NO: 1;
b. a nucleotide sequence with at least 90% identity to nt position 38 to nt position 1084 of SEQ ID NO: 1;
c. a nucleotide sequence encoding a protein having the amino acid sequence of SEQ ID NO: 2, 3, 4, or 5;
d. a nucleotide sequence encoding an allelic variant or a fragment of SEQ ID NO: 2; and
e. a nucleotide sequence encoding a protein with at least 90% identity to SEQ ID NO: 2;
and growing a plant from said plant cell.

Item 8. The method of Item 7, further comprising selecting a plant with improved drought resistance as compared to a control plant.

Item 9. The method of Item 7, wherein said plant is selected from the group consisting of corn, soybean, cotton, canola, rice, barley, oats, wheat, turf grass, alfalfa, sugar beet, sunflower, quinoa and sugar cane.

Item 10. A DNA construct comprising a polynucleotide encoding a polypeptide, wherein the nucleotide sequence of the polynucleotide is selected from the group consisting of:
a. a nucleotide sequence set forth as SEQ ID NO: 1, or nt position 38 to nt position 1084 of SEQ ID NO: 1;
b. a nucleotide sequence with at least 90% identity to nt position 38 to nt position 1084 of SEQ ID NO: 1;
c. a nucleotide sequence encoding a protein having the amino acid sequence of SEQ ID NO: 2, 3, 4, or 5;
d. a nucleotide sequence encoding an allelic variant or a fragment of SEQ ID NO: 2; and
e. a nucleotide sequence encoding a protein with at least 90% identity to SEQ ID NO: 2

### DETAILED DESCRIPTION OF THE INVENTION

Unless specifically defined, all technical and scientific terms used herein have the same meaning as commonly understood by persons of ordinary skill in the art. The procedures for preparing and screening transgenic plants described below are well known and commonly employed by persons of ordinary skill in the art.

The OsPPI8 gene of the present invention is useful in genetically improving drought resistance of a plant such as rice. The gene can be engineered into a vector to form a DNA construct. The DNA construct can be used in transformation of a plant cell or tissue of a plant such as rice to produce a transgenic plant cell and/or transgenic plant. In the transgenic plant cell and transgenic plant, the OsPP18 gene of the present invention is expressed in an increased level which confers increase level of drought resistance. Therefore, the present invention also relates to a method of improving drought resistance of a plant, wherein said plant is subjected to a treatment so that the OsPP18 gene of the present invention is expressed at an increased level in said plant in comparison with an identical control plant without the treatment. The OsPPI8 gene, DNA construct comprising said gene, plant cells or plants comprising transformed OsPPI8 gene and use and method of use of the gene of the present invention for improving drought resistance of a plant such as rice are all encompassed in the present invention.

The terms “fragment” or “fragment of a sequence” mean a truncated sequence of the original sequence referred to. The truncated sequence (nucleic acid or protein sequence) can vary widely in length; the minimum size being a sequence of sufficient size to provide a sequence with at least a comparable function and/or activity of the original sequence referred to.

The term “homolog” means a protein in a group of proteins that perform the same biological function, e.g. proteins that belong to the same Pfam protein family and that provide a common enhanced trait in transgenic plants of this disclosure. Homologs are expressed by homologous genes. With reference to homologous genes, homologs include orthologs, e.g., genes expressed in different species that evolved from a common ancestral genes by speciation and encode proteins retain the same function, but do not include paralogs, e.g., genes that are related by duplication but have evolved to encode proteins with different functions. Homologous genes include naturally occurring alleles and artificially-created variants. Degeneracy of the genetic code provides the possibility to substitute at least one base of the protein encoding sequence of a gene with a different base without causing the amino acid sequence of the polypeptide produced from the gene to be changed. When optimally aligned, homolog proteins have typically at least about 60% identity, in some instances at least about 70%, at least about 75%, at least about bout 80%, about 85%, at least about 90%, at least about bout 92%, at least about bout 94%, at least about bout 95%, at least about 96%, at least about 97%, at least about 98%, at least about 99%, and even at least about 99.5% identity over the full length of a protein identified as being associated with imparting an enhanced trait when expressed in plant cells. In one aspect of the disclosure homolog proteins have an amino acid sequences that have at least about 80%, at least about 85%, at least about 90%, at least about 92%, at least about 94%, at least about 95%, at least about 96%, at least about 97%, at least about 98%, at least about 99%, and at least about 99.5% identity to a consensus amino acid sequence of proteins and homologs that can be built from sequences disclosed herein.

Homologs can be identified by comparison of amino acid sequence, e.g. manually or by use of a computer-based tool using known homology-based search algorithms such as those commonly known and referred to as BLAST, FASTA, and Smith-Waterman. A local sequence alignment program, e.g. BLAST, can be used to search a database of sequences to find similar sequences, and the summary Expectation value (E-value) used to measure the sequence base similarity. A further aspect of the homologs encoded by DNA useful in the transgenic plants of the disclosure are those proteins that differ from a disclosed protein as the result of deletion or insertion of one or more amino acids in a native sequence.

Other functional homolog proteins differ in one or more amino acids from those of a trait-improving protein disclosed herein as the result of one or more of the well-known conservative amino acid substitutions, e.g., valine is a conservative substitute for alanine and threonine is a conservative substitute for serine. Conservative substitutions for an amino acid within the native sequence can be selected from other members of a class to which the naturally occurring amino acid belongs. Representative amino acids within these various classes include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, tyrosine, asparagine, and glutamine; and (4) neutral nonpolar (hydrophobic) amino acids such as alanine, leucine, isoleucine, valine, proline, phenylalanine, tryptophan, and methionine. Conserved substitutes for an amino acid within a native protein or polypeptide can be selected from other members of the group to which the naturally occurring amino acid belongs. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side 30 chains is cysteine and methionine. Naturally conservative amino acids substitution groups are: valine-leucine, valine-isoleucine, phenylalanine-tyrosine, lysine-arginine, alaninevaline, aspartic acid-glutamic acid, and asparagine-glutamine. A further aspect of the disclosure includes proteins that differ in one or more amino acids from those of a described protein sequence as the result of deletion or insertion of one or more amino acids in a native sequence.

A gene or protein often has different allelic variants in nature that maintain the same function and activities of the gene or protein. Such natural alleles of OsPP18 gene and corresponding protein are also encompassed within the present invention. Allelic variants encompass Single Nucleotide Polymorphisms (SNPs), as well as Small Insertion/Deletion Polymorphisms (INDELs). The size of INDELs is usually less than 100 bp.

A transgenic “plant cell” means a plant cell that is transformed with stably-integrated, non-natural, recombinant polynucleotides, e.g. by Agrobacterium-mediated transformation or by bombardment using micropaiticles coated with recombinant polynucleotides. A plant cell of this invention can be an originally-transformed plant cell that exists as a microorganism or as a progeny plant cell that is regenerated into differentiated tissue, e.g. into a transgenic plant with stably-integrated, non-natural recombinant polynucleotides in its chromosomal DNA, or seed or pollen derived from a progeny transgenic plant.

A “transgenic” plant or seed means one whose genome has been altered by the stable incorporation of recombinant polynucleotides in its chromosomal DNA, e.g. by transformation, by regeneration from a transformed plant from seed or propagule or by breeding with a transformed plant. Thus, transgenic plants include progeny plants of an original plant derived from a transformation process including progeny of breeding transgenic plants with wild type plants or other transgenic plants. The enhancement of a desired trait can be measured by comparing the trait property in a transgenic plant which has recombinant DNA conferring the trait to the trait level in a progenitor plant.

“Gene expression” means the function of a cell to transcribe recombinant DNA to mRNA and translate the mRNA to a protein. For expression the recombinant DNA usually includes regulatory elements including 5′ regulatory elements such as promoters, enhancers, and introns; other elements can include polyadenylation sites, transit peptide DNA, markers and other elements commonly used by those skilled in the art. Promoters can be modulated by proteins such as transcription factors and by intron or enhancer elements linked to the promoter.

“An increased level” of expression means an increase in the gene expression that is helpful for the drought resistance of the plant, e.g., at least about 5%, at least about 10%, at least about 15%, at least about 20%, at least about 25%, at least about 30%, at least about 40% at least about 50%, at least about 100%, at least about 200% increase in comparison with an identical control without the treatment of the present invention.

“Recombinant polynucleotide” means a DNA construct that is made by combination of two otherwise separated segments of DNA, e.g., by chemical synthesis or by the manipulation of isolated segments of nucleic acids by genetic engineering techniques. Recombinant DNA can include exogenous DNA or simply a manipulated native DNA. Recombinant DNA for expressing a protein in a plant is typically provided as an expression cassette which has a promoter that is active in plant cells operably linked to DNA encoding a protein, linked to a 3′ DNA element for providing a polyadenylation site and signal. Useful recombinant DNA also includes expression cassettes for expressing one or more proteins conferring stress tolerance.

Recombinant DNA constructs generally include a 3″ element that typically contains a polyadenylation signal and site. Well-known 3′ elements include those from Agrobacterium tumefaciens genes such as nos 3′, tml 3′, ttnr 3′, tms 3′, ocs 3\ tr73′, e.g., disclosed in U.S. Pat. No. 6,090,627. 3′ elements from plant genes such as a rice glutelin gene, a rice lactate dehydrogenase gene and a rice beta-tubulin gene are disclosed in U.S. published patent application 2002/0192813 A1.

The expression construct comprising OsPP18 gene according to the present invention can be introduced into the plant cells by conventional biological techniques, such as T1 plasmid, plant virus vector, direct DNA transformation, microinjection, electroporation, etc. (Weissbach, 1998, Method for Plant Molecular Biology VIII, Academy Press, new York, pp. 411-463; Geiserson and Corey, 1998, Plant Molecular Biology (2^{nd} Edition)).

The expression vectors comprising the OsPPI8 gene of the present invention can be transformed into multiple host plants including rice to breed plant varieties with drought resistance.

The hosts, into which the expression construct comprising OsPP18 gene according to the present invention may be many plants, especially cultivated varieties, including rice.

Since the expression of the gene of the present invention is induced by drought stress, the gene of the present invention can be combined with any drought-inducible promoter of interest, and then be inserted into a suitable expression carrier. After being transformed into the plant host, the gene can be expressed under the inducement of drought condition, therefore enhancing the drought resistance of the plant.

Plant Cell Transformation Methods

Numerous methods for transforming plant cells with recombinant DNA are known in the art and may be used in the present invention. Two commonly used methods for plant transformation are Agrobacterium-mediated transformation and microprojectile bombardment. Microprojectile bombardment methods are illustrated in U.S. Pat. No. 5,015,580 (soybean); U.S. Pat. No. 5,550,318 (corn); U.S. Pat. No. 5,538,880 (corn); U.S. Pat. No. 5,914,451 (soybean); U.S. Pat. No. 6,160,208 (corn); U.S. Pat. No. 6,399,861 (corn) and U.S. Pat. No. 6,153,812 (wheat) and Agrobacterium-mediated transformation is described in U.S. Pat. No. 5,159,135 (cotton); U.S. Pat. No. 5,824,877 (soybean); U.S. Pat. No. 5,591,616 (corn); and U.S. Pat. No. 6,384,301 (soybean), all of which are incorporated herein by reference. For Agrobacterium tumefaciens based plant transformation system, additional elements present on transformation constructs will include T-DNA left and right border sequences to facilitate incorporation of the recombinant polynucleotide into the plant genome.

In general it is useful to introduce recombinant DNA randomly, i.e. at a non-specific location, in the genome of a target plant line. In special cases it may be useful to target recombinant DNA insertion in order to achieve site-specific integration, for example to replace an existing gene in the genome, to use an existing promoter in the plant genome, or to insert a recombinant polynucleotide at a predetermined site known to be active for gene expression. Several site specific recombination systems exist which are known to function implants include cre-Iox as disclosed in U.S. Pat. No. 4,959,317 and FLP-FRT as disclosed in U.S. Pat. No. 5,527,695.

Transformation methods of this invention are preferably practiced in tissue culture on media and in a controlled environment. “Media” refers to the numerous nutrient mixtures that are used to grow cells in vitro, that is, outside of the intact living organism. Recipient cell targets include, but are not limited to, meristem cells, callus, immature embryos and gametic cells such as microspores, pollen, sperm and egg cells. It is contemplated that any cell from which a fertile plant may be regenerated is useful as a recipient cell. Callus may be initiated from tissue sources including, but not limited to, immature embryos, seedling apical meristems, microspores and the like. Cells capable of proliferating as callus are also recipient cells for genetic transformation. Practical transformation methods and materials for making transgenic plants of this invention, for example various media and recipient target cells, transformation of immature embryo cells and subsequent regeneration of fertile transgenic plants are disclosed in U.S. Pat. Nos. 6,194,636 and 6,232,526, which are incorporated herein by reference.

The seeds of transgenic plants can be harvested from fertile transgenic plants and be used to grow progeny generations of transformed plants of this invention including hybrid plants line for selection of plants having an enhanced trait. In addition to direct transformation of a plant with a recombinant DNA, transgenic plants can be prepared by crossing a first plant having a recombinant DNA with a second plant lacking the DNA. For example, recombinant DNA can be introduced into first plant line that is amenable to transformation to produce a transgenic plant which can be crossed with a second plant line to introgress the recombinant DNA into the second plant line. A transgenic plant with recombinant DNA providing an enhanced trait, e.g. enhanced yield, can be crossed with transgenic plant line having other recombinant DNA that confers another trait, for example drough resistance or pest resistance, to produce progeny plants having recombinant DNA that confers both traits.

In the practice of transformation DNA is typically introduced into only a small percentage of target plant cells in any one transformation experiment. Marker genes are used to provide an efficient system for identification of those cells that are stably transformed by receiving and integrating a transgenic DNA construct into their genomes. Preferred marker genes provide selective markers which confer resistance to a selective agent, such as an antibiotic or herbicide. Any of the herbicides to which plants of this invention may be resistant are useful agents for selective markers. Potentially transformed cells are exposed to the selective agent. In the population of surviving cells will be those cells where, generally, the resistance-conferring gene is integrated and expressed at sufficient levels to permit cell survival. Cells may be tested further to confirm stable integration of the exogenous DNA. Commonly used selective marker genes include those conferring resistance to antibiotics such as kanamycin and paromomycin (nptll), hygromycin B (aph IV) and gentamycin (aac3 and aacCA) or resistance to herbicides such as glufosinate (bar or pat) and glyphosate (aroA or EPSPS). Examples of such selectable are illustrated in U.S. Pat. Nos. 5,550,318; 5,633,435; 5,780,708 and 6,118,047. Selectable markers which provide an ability to visually identify transfonnants can also be employed, for example, a gene expressing a colored or fluorescent protein such as a luciferase or green fluorescent protein (GFP) or a gene expressing a fteto-glucuronidase or uidA gene (GUS) for which various chromogenic substrates are known.

Plant cells that survive exposure to the selective agent, or plant cells that have been scored positive in a screening assay, may be cultured in regeneration media and allowed to mature into plants. Developing plantlets regenerated from transformed plant cells can be transferred to plant growth mix, and hardened off, for example, in an environmentally controlled chamber at about 85% relative humidity, 600 ppm CO₂, and 25-250 microeinsteins m⁻¹s⁻¹ of light, prior to transfer to a greenhouse or growth chamber for maturation. Plants are regenerated from about 6 weeks to 10 months after a transformant is identified, depending on the initial tissue. Plants may be pollinated using conventional plant breeding methods known to those of skill in the art and seed produced, for example self-pollination is commonly used with transgenic corn. The regenerated transformed plant or its progeny seed or plants can be tested for expression of the recombinant DNA and selected for the presence of enhanced agronomic trait.

Transgenic Plants and Seeds

Transgenic plants derived from the plant cells of this invention are grown to generate transgenic plants having an enhanced trait as compared to a control plant and produce transgenic seed and haploid pollen of this invention. Such plants with enhanced traits are identified by selection of transformed plants or progeny seed for the enhanced trait. For efficiency a selection method is designed to evaluate multiple transgenic plants (events) including the recombinant DNA, for example multiple plants from 2 to 20 or more transgenic events. Transgenic plants grown from transgenic seed provided herein demonstrate improved agronomic traits that contribute to increased yield or enhanced water deficit tolerance or both.

The further illustration of the present invention is given below in reference to the figures and examples.

### DESCRIPTION OF THE DRAWINGS

SEQ ID No: 1 is the nucleotide acid sequence that contains OsPP18 gene which is isolated and cloned in the present invention. The length of the sequence is 1162 bp. The protein coding region lies between the nucleotide acids from position 38 to position 1084, encoding 348 amino acids.

SEQ ID NO: 2 is the protein sequence of OsPP18 gene.

SEQ ID NO: 3, 4, and 5 are the homologs of SEQ ID NO: 2.

FIG. 1. The expression of OsPP18 gene in ospp18 mutant. WT is the negative control from the same rice variety as the transgenic plants. ospp18 is OsPP18 mutant generated by T-DNA insertion.

FIG. 2. The expression pattern of OsPP18 gene under various adverse situations and hormone treatments. The samples are treated as follows: drought treatment for 0 d, 3 d, 5 d, 7 d, rehydrate 1 d; salt treatment for 0 h, 1 h, 3 h, 6 h, 12 h, 24 h; cold treatment for 0 h, 1 h, 3 h, 6 h, 12 h, 24 h; heat treatment for 0 min, 10 min, 30 min, 1 h, 3 h, 6 h; oxidative stress (H₂O₂) treatment for 0 h, 1 h, 3 h, 6 h, 12 h, 24 h; UV treatment for 0 h, 3 h, 6 h, 12 h; wound treatment for 0 h, 1 h, 3 h, 6 h; submerge stress treatment for 0 h, 6 h, 12 h, 24 h, 72 h. Hormone treatments: abscisic acid (ABA), jasmonic acid (JA), salicylic acid (SA) treatments for 0 h, 1 h, 3 h, 6 h, 12 h.

FIG. 3. Phenotypes of rice ospp18 mutants at seedling stage under drought stress and the survival statistics of the ospp18 mutants at seedling stage under drought stress. A: Phenotypes of rice ospp18 mutants at seedling stage under drought stress. ospp18#1, #2, and #3 are homozygous OsPP18 mutant lines generated by T-DNA insertion. WT#1, #2, and #3 are three negative lines isolated from the heterozygous mutants. B is the statistic results of the survival rate of the ospp18 mutants at seedling stage under drought stress. ospp18#1, #2, and #3 are three homozygous OsPP18 mutant lines. WT#1, #2, and #3 are three negative lines isolated from the heterozygous mutants.

FIG. 4. Phenotypes of rice ospp18 mutants under drought stress at adult plant stage in the field, the statistics of the biomass of the ospp18 mutants after drought stress at adult plant stage in the field, the statistics of the plant height of the ospp18 mutants under drought stress at adult plant stage in the field, and the statistics of the seed setting rate of the ospp18 mutants under drought stress at adult plant stage in the field. A: Phenotypes of rice ospp18 mutants under drought stress at adult plant stage in the field. ospp18 is homozygous mutant. WT is isolated negative control. B: the statistics of the biomass of the ospp18 mutants under drought stress at adult plant stage in the field. ospp18 is homozygous mutant. WT is isolated negative lines. C: the statistics of the plant height of the ospp18 mutants under drought stress at adult plant stage in the field. ospp18 is homozygous mutant. WT is isolated negative control. D: the statistics of the seed setting rate of the ospp18 mutants under drought stress at adult plant stage in the field. ospp18 is homozygous mutant. WT is isolated negative lines.

FIG. 5. Phenotypes of rice ospp18 mutants under osmosis stress and the statistics of the plant height of the ospp18 mutants under osmosis stress. A: Phenotypes of rice ospp18 mutants under osmosis stress. ospp18 is homozygous mutant. WT is isolated negative control. B: the statistic results of the plant height of the ospp18 mutants under osmosis stress. ospp18 is homozygous mutant. WT is isolated negative lines.

FIG. 6. Schematic picture of the OsPPJ8 overexpression construct, the expression pattern of OsPP18 gene in OsPP18 overexpressing plants, phenotypes of OsPPJ8 overexpressing plants under osmosis stress, and the statistic results of the plant height of the OsPP18 overexpressing plants under osmosis stress. A: Schematic picture of the OsPP18 overexpression construct. B: the expression of OsPP18 gene in OsPP18 overexpressing plants. “Zhonghua 11” (ZH11) is used as a wild type line. C: phenotypes of OsPP18 overexpressing plants under osmosis stress. OsPP18-OX-4 and -7 are T1 generation of two independent transgenic lines with overexpression. ZH11 is a wild type line. D: the statistic results of the plant height of the OsPP18 overexpressing plants under osmosis stress. OsPP18-OX-4 and -7 are T1 generation of two independent transgenic lines with overexpression. ZH11 is a wild type line.

FIG. 7. Plasmid profile of pCAMBIA1301 vector.

FIG. 8. Plasmid profile of pU1301 vector and OsPP18 overexpression construct.

### EXAMPLES

The following examples describe the methods for isolating OsPP18 T-DNA insertion mutant, cloning the DNA fragment that comprises the entire coding region of OsPP18 gene, and verifying the function of OsPP18 gene. According to the following description and the examples, those skilled in the art can determine the basic technical features of the present invention, and can make various changes and modifications to the present invention in order to adapt it to various uses and conditions without departing from the spirit and scope of the present invention. Therefore, all matter set forth or shown in the accompanying drawings and examples is to be interpreted as illustrative and not in a limiting sense.

2. Isolation of OsPP18 T-DNA Mutant

The T-DNA insertion mutant 2D-21407 in which the insertion occurs on the locus of OsPPJ8 gene was selected from the Rice T-DNA Insertion Sequence Database (RISD) (The starting material of the present invention is mutant 2D-21407, the website is http://signal.salk.edu/cgi-bin/RiceGE administered by Plant Functional Genomics Laboratory of Korea). The methods of constructing this mutant vector and genetic transformation are described in the related literatures (Jeong et al., Generation of a flanking sequence-tag database for activation-tagging lines in japonica rice. Plant J. 2006, 45:123-32), with respect to which the present description will not provide more descriptions in detail. In the above mentioned mutant database, the sequence (with 480 bp in length) flanking the OsPP18 T-DNA mutant 2D-21407 is set forth as follows:

GNNATNCCCCAGTTGTCATGTATTAGCCACATAGCAAAAAAAATAGCACC
GTGGTAGTAAGAATGGAACTCACCTGGTACCTGGTACCTCGGATCCGTGT
TTTCGTGTTAGGGGGAAGATCCAAGAGGAGGAGGAGGAGGAGGAGAAGGA
TGACAAGTTTTTCATGGCGCGGTCAGGGGCTTTGTGCTTGATTTGATTGG
CGGCTAACTATCGAGTAGTGATTCTTGAATCAGAGGCTAGGACATGAGGT
TTCAGTGGCGGATTTTGAATTAGATCTGCCCGATTTTGCTCTGAATTAGC
TTAGCCTAATCGGGTTCCAGCTCCTGGCTGGTGTGAGGATGTGGCCATGA
TTTTGAATTTGAGGAGTGTTTGAAAACNAAAAGTCTAATCCTTTGTAAGG
GCATATGGTANAGGAATTAACCTTTTTCTTGTTTGATGTGTCTGTGTGNA
NCANCTGATTTTTATGGANTGTTNTTANTT

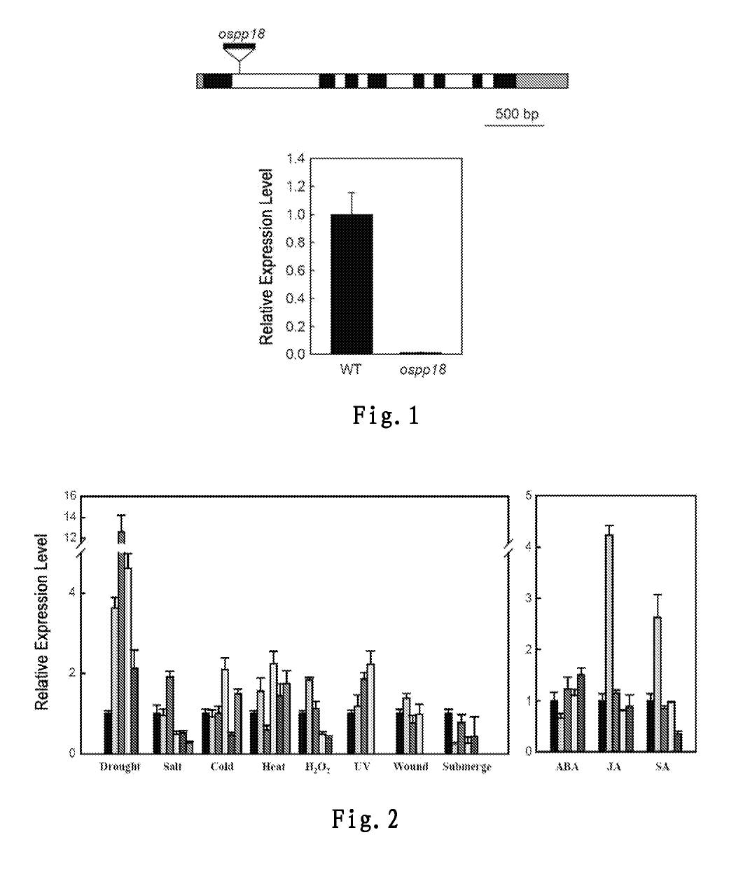

According to the insertion site of T-DNA, primers were designed to measure the expression level of OsPP18 gene in the mutant. PCR primers (OsPP 18-F: 5′-CACAAACCCGACCAGTCAGA-3′ and OsPP18-R: 5′-CGTCCCAGCCCACATCA-3′) were used to specifically amplify OsPP18 gene. Meanwhile, a 76 bp fragment of the rice Actin1 gene was specifically amplified with primers (AF: 5′-TGGCATCTCTCAGCACATTCC-3′ and AR 5′-TGCACAATGGATGGGTCAGA-3′) as an internal control for quantitative analysis. The PCR protocol was: 95° C. 10 sec; 95° C. 5 sec, 60° C. 34 sec, 40 cycles. Fluorescent quantitative analysis was conducted in real time during the PCR process. The results demonstrated that: the expression of OsPP18 gene in the homozygous ospp18 T-DNA insertion mutant was significantly lower than that that in the isolated negative control (FIG. 1). Thus, OsPP18 gene is significantly suppressed in the ospp18 mutant.

3. Determination of the Expression Level of the Endogenous Rice OsPP18 Gene.

Applicants selected the japonica rice variety “Zhonghua 11” (developed by the Institute of Crop Sciences of Chinese Academy of Agricultural Sciences as a publicly popularized rice variety) as the material to study the expression pattern. The 4-leaf seedlings originating from sprouting seeds under normal growth conditions were treated under various stress conditions or hormones. The drought treatment was conducted by directly exposing the seedlings in air without watering to dehydrate for 0 d, 3 d, 5 d, 7 d followed by rehydrate for 1 d before sampling. The high-salinity stress was conducted by soaking the seedlings in 200 mmol/L NaCl solution for 0 h, 1 h, 3 h, 6 h, 12 h, 24 h before sampling. The cold stress was conducted by placing the seedlings in a 4° C. growth chamber for 0 h, 1 h, 3 h, 6 h, 12 h, 24 h before sampling. The heat stress was conducted by placing the seedlings in a 42° C. growth chamber for 0 min, 10 min, 30 min, 1 h, 3 h, 6 h before sampling. UV treatment was conducted by placing the seedlings under a UV lamp for 0 h, 3 h, 6 h, 12 h before sampling. The wounding treatment was conducted by mechanically wounding the seedlings using forceps and sampling after 0 h, 1 h, 3 h, 6 h. Oxidative stress was conducted by soaking the seedlings in 1% H₂O₂ solution for 0 h, 1 h, 3 h, 6 h, 12 h before sampling. The submerge stress was conducted by submerging the seedlings in a transparent container filled with water for 0 h, 6 h, 12 h, 24 h, 72 h before sampling. Hormone treatment was conducted by evenly spraying the surface of the plants with 100 μmol/L abscisic acid (ABA), jasmonic acid (JA), or salicylic acid (SA), respectively; leaving the hormone solution to the root of the seedlings; and sampling after 0 h, 1 h, 3 h, 6 h, 12 h. Total rice RNA was extracted using TRIZOL reagent (from Invitrogen Co.) according to the specification of the manufacturer and reverse-transcribed into eDNA using reverse transcriptase SSIII (from Invitrogen Co.) according to the specification of the manufacturer. The reaction was conducted as follows: 65° C. 5 min, 50° C. 120 min, 70° C. 10 min. With the above reverse transcribed cDNA as template, OsPP18 gene was specifically PCR amplified using primers (OsPP18-F: 5′-CACAAACCCGACCAGTCAGA -3′ and OsPP18-R: 5′-CGTCCCAGCCCACATCA-3′). Meanwhile, a 76 by fragment of the rice Actin1 gene was specifically amplified with primers (AF: 5′-TGGCATCTCTCAGCACATTCC-3′ and AR: 5′-TGCACAAT GGATGGGTCAGA-3′) as internal control for quantitative analysis. PCR reaction was conducted as follows: 95° C. 10 sec; 95° C. 5 sec, 60° C. 34 sec, 40 cycles. Fluorescent quantitative analysis was conducted in real time during the reaction process. The results (FIG. 2) demonstrated that the expression level of OsPP18 gene (SEQ NO:1) was enhanced upon the induction of drought, high-salinity, cold, heat, UV, and oxidative stresses. OsPP18 gene was rapidly induced by jasmonic acid and salicylic acid, but was not induced by ABA.

4. Identification of the Phenotype of Ospp18 Mutant Under Drought Stress.

The sprouting seeds of genotyped homozygous mutant (ospp18) and wild type line (WT) were seeded in small round buckets. The soil used in the experiments was a mixture of South China rice soil and sand at the ratio of 2:3. Homogenous soil/sand mixture and same volume of water were added into each bucket. Water was allowed to drain naturally so as to ensure the consistency of soil compactness. The experiment was in triplet. The healthy plants in 4-leaf stage were treated with drought stress for 6-10 days (depending on the particular weather), then were rehydrated for 5-7 days. Pictures were taken and survival rate was recorded. Compared with the wild type control, T-DNA homozygous plants demonstrated a drought-sensitive phenotype.

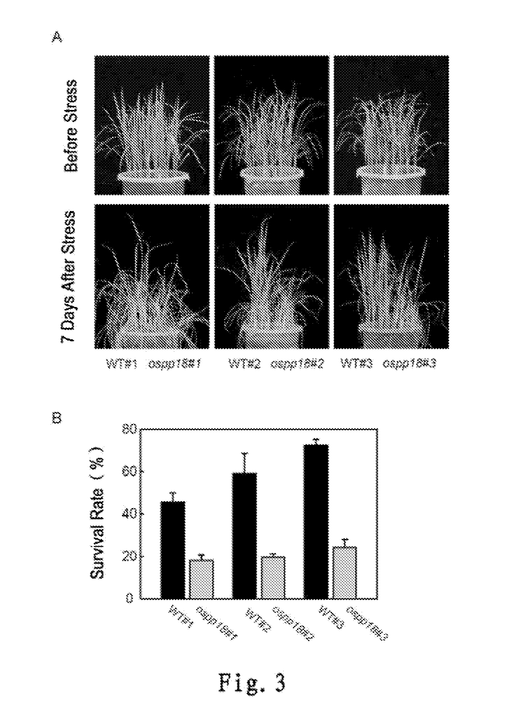

To investigate the co-segregation of ospp18 mutant and the drought-sensitive phenotype, the seeds from individual T1 heterozygous plants were collected and seeded to obtain T2 homozygous plants and wild type plants. Seeds were collected from the T2 plants and were developed into lines. Three homozygous mutant lines (ospp18#1, #2, #3) and three isolated wild type lines (WT#1, #2, #3) were randomly selected and were subject to the same afore-described stress experiments. Homozygous lines were more sensitive to drought as compared with the negative controls (FIG. 3. A). After rewatering, the survival rates of the homozygous lines were lower than 25%, while the survival rates of the wild type lines were higher than 40% (FIG. 3. B). The experiments were repeated for three times and rendered the same results, showing that the phenotype of the mutant was indeed caused by the insertion of T-DNA.

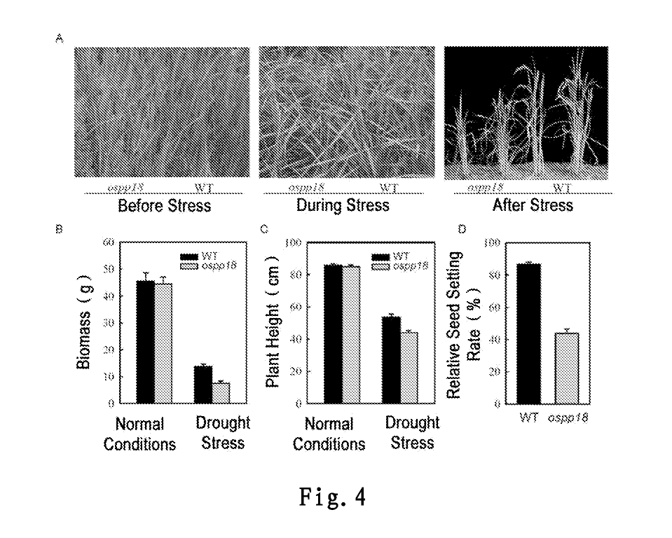

To investigate the phenotype of the mutant at adult plant stage, mutant and control lines were planted in a field covered by removable rainproof shed, which comprises Southern China rice soil and sand at a ratio of 1:2. Each line was planted in two rows with 10 plants per row. Severe drought stress experiment was performed in triplet. To mimic drought stress, healthy plants at adult plant stage were left without watering for 15-20 days (depending on the particular weather, the removable shed was used to shield the rain), and then were rehydrated. In comparison with the wild type controls isolated from the heterozygous parent line, the growth of the homozygous lines was obviously suppressed, demonstrating drought-sensitive phenotype (FIG. 4. A). The studies of the plant height and the seed setting rate showed that, the above-ground biomass (FIG. 4. B) and the plant height (FIG. 4. C) of the mutant under drought stress condition at adult plant stage were significantly lower than those of the control. Since the mutant and the control have different seed setting rates even under normal conditions, relative seed setting rate (the seed setting rate under drought stress divided by the seed setting rate under normal conditions) was used to compare the two lines. The result indicated that the relative seed setting rate of the mutant is significantly lower than that of the control (FIG. 4. D).

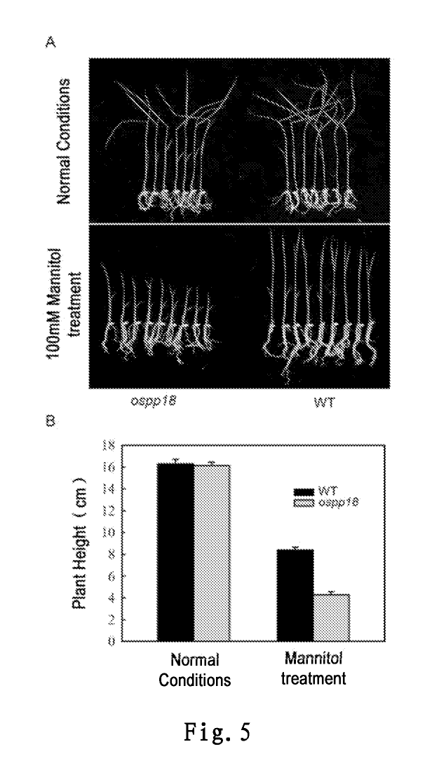

To investigate the phenotype of the mutant under osmosis stress, the mutant and control, after sprouting, were placed in ½ MS growing medium containing 100 mM mannitol (forming an environment with high solute concentration to mimic the osmosis stress). As a result, the growth of the mutant was obviously weaker than that of the control (FIG. 5. A). Measurement of the plant height indicated that the plant height of the mutant was significantly lower than that of the control (FIG. 5. B).

4. Construction and Genetic Transformation of the Overexpression Vector of OsPP18 Gene.

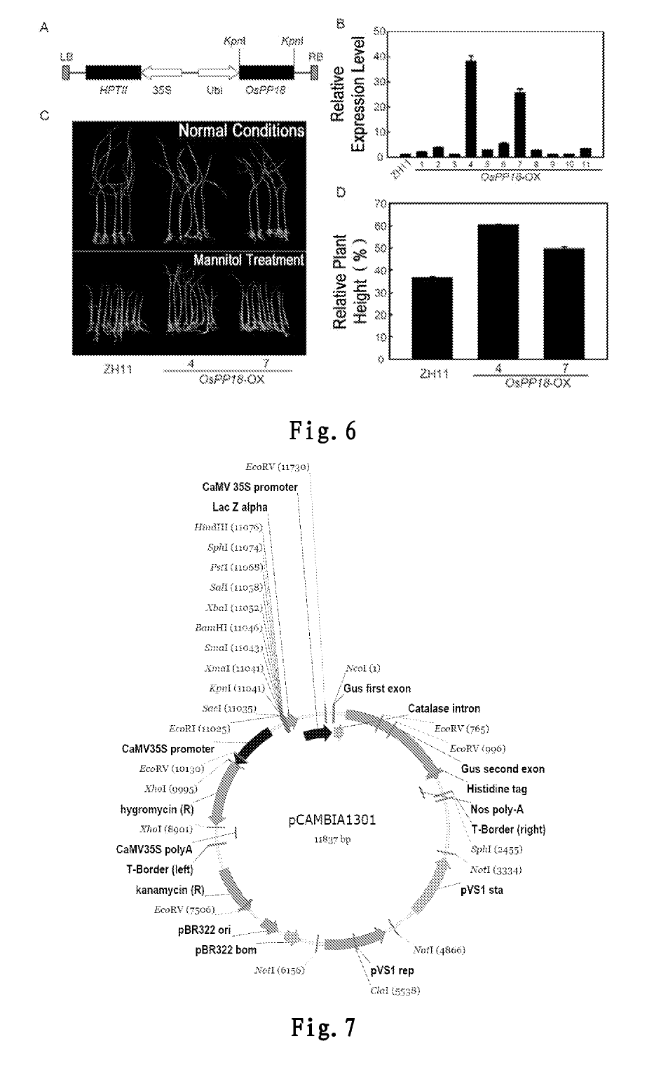

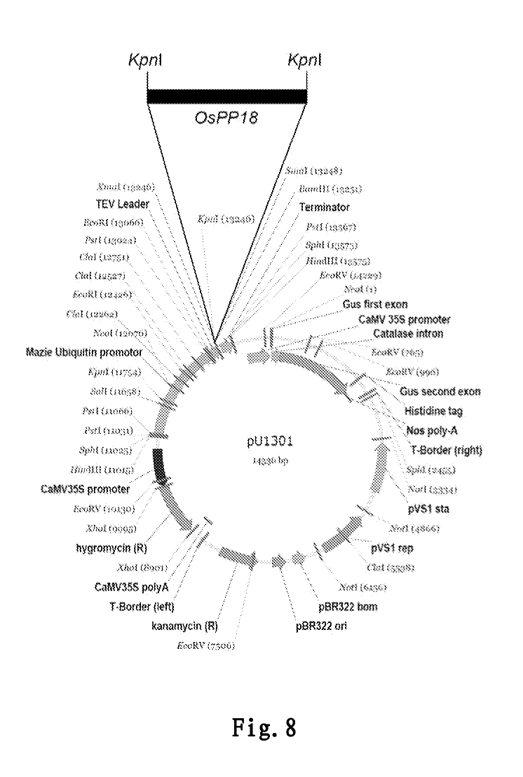

In order to better understand the function of OsPP18 gene, the applicants overexpressed the gene in rice and investigated the phenotypes of the transgenic plants. The overexpression vector was constructed as follows: firstly, on the website of Rice Genome Annotation, RGAP (http://rice.plantbiology.msu.edu/), the Annotation No. of OsPP18 gene is LOC_Os02g05630. While on KOME (http://cdna01.dna.affrc.go.jp/cDNA/), the Annotation No. of OsPP18 is AK066016. Database search indicated that OsPP18 is a member of the PP2C family. According to the sequence of the gene, PCR primers were designed. The total RNA of the callus of “Zhonghan 5” (a commercial rice variety provided by Shanghai Academy of Agricultural Sciences, China) was reversely transcribed to obtain cDNAs. With said cDNAs as template, using primers OSPP18FLF (5′-ATCGGTACCCTCCTCCATCCATTCCC-3′, sequence-specific primer with additional KpnI cleavage site) and OSPP18FLR (5′-ATCGGTACCGGTGCCGCCACTGTAA-3′, sequence-specific primer with additional BamHI cleavage site), a cDNA fragment comprising the complete coding region of OsPP18 gene was amplified. The sequence of the PCR product is set forth in SEQ ID NO: 1 (I-1162 bp). PCR reaction was conducted as follows: predenaturation at 94° C. for 5 min; 94° C. for 30 sec, 58° C. for 30 sec, 72° C. for 70 sec, 32 cycles; and elongation at 72° C. for 5 min. The PCR product obtained by amplification was ligated into pGEM-T vector (from Promega Co.) and the positive clone was screened and sequenced, resulting in the desired full length gene. The positive clone was named PGEM-OsPP18. Then, the positive clone plasmid PGEM-OsPP18 was enzymatically cleaved with KpnI, and the exogenous fragments were recovered. Meanwhile, the genetic transformation vector pU1301 with the ubiquitin promoter was enzymatically cleaved in the same way. pU1301 was reconstructed based on the genetic transformation vector pCAMBIA1301 (FIG. 7) that is commonly used internationally. It is an Agrobacterium-mediated vegetable genetic transformation vector carrying corn ubiquitin promoter with constitutive and overexpression characteristics (schematic picture shown in FIG. 8). After cleavage, the products of enzymatical cleavage was extracted and purified with chloroform: isopentanol (24:1 v/v). The digested OsPP18 gene fragment was ligated with the digested pCAMBIA1301U vector. Ligation product was then transformed into E. coli DH10β (the E. coli DH10β strain was purchased from Invitrogen Co.). Positive clones were verified by enzymatic digestion. The resulted recombination vector was named as OsPP18-OX-pU1301 (wherein the OsPP18 gene has the nucleotide sequence as set forth in SEQ ID NO:1 with 1162 bp in length, see FIG. 6 A).

By means of Agrobacterium mediated rice genetic transformation (described as follows), the above overexpression vector OsPP18-OX-pU1301 was transformed into the rice variety “Zhonghua 11” (from Institute of Crop Science, Chinese Academy of Agricultural Sciences). Transgenic plants were then obtained by precultivation, infestation, co-culture, screening for callus with hygromycin resistance, differentiation, rooting, seedling training and transplanting. The above Agrobacterium mediated rice (Zhonghua 11) genetic transformation method (system) was modified based on the method reported by Hiei, et al (Hiei, et al., Efficient transformation of rice, Oryza saliva L., mediated by Agrobacterium and sequence analysis of the boundaries of the T-DNA, Plant J, 6:271-282, 1994).

The particular procedure of the genetic transformation in the present example is as follows:

(1) Electrotransfonnation: The resulted overexpression vector OsPP18-OX-pU1301 (see FIG. 8. for plasmid profile) was electrotransformed into the Agrobacterium strain EHA105 under an 1800V electropulse. The electrotransformed cells were then spread on regular selective LA medium having corresponding selectivity. Positive clone was screened and prepared for following infection of callus.

(2) Callus Induction: Mature rice seeds “Zhonghua 11” were deshelled, then treated with 70% alcohol for 1 minute and disinfected on the surface by 0.15% HgCl₂ for 15 minutes. The seeds were washed with sterilized water for 4-5 times. The sterilized seeds were put on the induction medium (described below). The inoculated callus induction medium (described below) was placed in darkness and cultured for 4 weeks at 25±1° C.

(3) Callus Subculture: The bright yellow, compact and relatively dry embryogenic callus was selected, put onto the subculture medium (described below), and cultured in darkness for 2 weeks at 25±1° C.

(4) Pre-culture: The compact and relatively dry rice embryogenic callus was selected, put onto the pre-culture medium (described below), and cultured in darkness for 2 weeks at 25±1° C.

(5) Agrobacterium Culture: Agrobacterium EHA105 (commercially available at CAMBIA Laboratory, Australia) transformed with the overexpression vector OsPP18-OX-pU1301 of the present invention was precultured on the selective LA medium at 28° C. for 2 days. Said Agrobacterium was transferred into suspension medium (described below), which was then put in swing bed at 28° C. for 2-3 hours.

(6) Agrobacterium Infection: The pre-cultured callus was transferred into a sterilized bottle. The Agrobacterium suspension was adjusted to OD₆₀₀ 0.8-1.0. The callus was immersed in the Agrobacterium suspension for 30 minute. The callus was transferred on sterilized filter paper and dried; and then cultured onto the cocultivation medium (described below) for 3 days at 19-20° C.

(7) Wash and Selective Culture of Callus: The callus was washed with sterilized water until no Agrobacterium was observed. The callus was immersed in sterilized water containing 400 ppm carbenicillin (CN) for 30 minutes. The callus was transferred on sterilized filter paper and dried. The callus was transferred on the selection medium (described below) and cultured and selected for 2-3 times, 2 weeks for each time (the concentration of carbenicillin was 400 ppm for the first culture, and 250 ppm for the second and subsequent cultures, the concentration of hygromycin was 250 ppm).

(8) Differentiation: The resistant calluses were transferred to the pre-differentiation medium (described below) and cultured in darkness for 5-7 weeks. The pre-differentiated calluses were then transferred to differentiation culture medium (described below) and cultured in lighting at 26° C.

(9) Rooting: The roots generated during the differentiation were cut off. Then the plant was transferred to the rooting culture medium and cultured in lighting at 26° C. for 2-3 weeks.

(10) Transplantation: The residual medium on roots of the plant was washed off; the seedlings with well-grown roots were transplanted into green house for growing; keeping the moisture in the beginning days.

The Compositions and Formulae of Culture Media:

(1) The abbreviations of the reagents and solutions: the abbreviations of the plant hormones used in the mediums of the present invention are: 6-BA (6-benzyladenine); CN (Carbenicillin); KT (Kinetin); NAA (Naphthalene acetic acid); IAA (Indole-3-acetic acid); 2,4-D (2,4-Dichlorphenoxyacetic acid); AS (Acetosringone); CH (Casein Enzymatic Hydrolysate); HN (Hygromycin B); DMSO (Dimethyl Sulfoxide); N6max (N6 solution with major elements); N6mix (N6 solution with trace elements); MSmax (MS solution with major elements); MSmix (MS solution with trace elements).

(2) the Formula of the Major Solutions:

1) Preparation of Concentrated Solution of N6 Medium with Major Elements (10× Concentrated Solution):

| | |
|---|---|
| Potassium nitrate (KNO₃) | 28.3 g |
| Monopotassium phosphate (KH₂PO₄) | 4.0 g |
| Ammonium sulfate ((NH₄)₂SO₄) | 4.63 g |
| Magnesium sulfate (MgSO₄•7H₂O) | 1.85 g |
| Calcium chloride (CaCl₂•2H₂O) | 1.66 g |

Dissolve them one by one and add water to the final volume 1000 ml at room temperature.

2) Preparation of Concentrated Solution of N6 Medium with Trace Elements (10× Concentrated Solution):

| | |
|---|---|
| Potassium iodide (KI) | 0.08 g |
| Boracic acid (H₃BO₃) | 0.16 g |
| Manganese sulfate (MnSO₄•4H₂O) | 0.44 g |
| Zinc sulfate (ZnSO₄•7H₂O) | 0.15 g |

Dissolve and add water to the final volume 1000 ml at room temperature.

3) Preparation of the Ferric Salt (Fe₂EDTA) Stock Solution (100×)

800 ml of double distilled water was heated to 70° C., 3.73 g of Disodium Ethylene Diamine Tetraacetic Acid (Na₂EDTA.2H2O) was added therein. After dissolving completely, the solution was kept in 70° water bath for 2 hours and water was added to a final volume 1000 ml and then the solution was kept at 4° C. for use.

4) Preparation of the Vitamin Stock Solution (100×)

| | |
|---|---|
| Nicotinic acid | 0.1 g |
| Vitamin B1 (Thiamine HCl) | 0.1 g |
| Vitamin B6 (Pyridoxine HCl) | 0.1 g |
| Glycine | 0.2 g |
| Inositol | 10 g |

Add water to a final volume 1000 ml and then keep the solution at 4° C. for use.

5) Preparation of the Concentrated Solution of MS Medium with Major Elements (10×):

| | |
|---|---|
| Ammonium nitrate (NH₄NO₃) | 16.5 g |
| Potassium nitrate | 19.0 g |
| Monopotassium phosphate | 1.7 g |
| Magnesium sulfate | 3.7 g |
| Calcium chloride | 4.4 g |

Dissolve the components and add water to the final volume 1000 ml at room temperature.

6) Preparation of the Concentrated Solution of MS Medium with Trace Elements (10×)

| | |
|---|---|
| Potassium iodide | 0.083 g |
| Boracic acid | 0.62 g |
| Manganese sulfate | 0.86 g |
| Sodium Molybdate (Na₂MoO₄•2H₂O) | 0.025 g |
| Copper sulphate (CuSO₄•5H₂O) | 0.0025 g |

Dissolve the components and add water to the final volume 1000 ml at room temperature.

7) 2,4-D stock solution, 6-BA stock solution, Naphthalene acetic acid (NAA) stock solution, and Indole-3-acetic acid (IAA) stock solution: all are 1 mg/ml.

8) Glucose stock solution: 0.5 g/ml.

9) Preparation of the AS stock solution: Weigh 0.392 g AS, dissolve completely in 10 ml DMSO.

(3) the Formulas of the Media for Rice Genetic Transformation

1) Callus Induction Medium

| | | |
|---|---|---|
| N6max concentrated solution (10X) | 100 | ml |
| N6mix concentrated solution (100X) | 10 | ml |
| Fe²⁺EDTA stock solution (100X) | 10 | ml |
| Vitamin stock solution (100X) | 10 | ml |
| 2,4-D stock solution | 2.5 | ml |
| Proline | 0.3 | g |
| CH | 0.6 | g |
| Sucrose | 30 | g |
| Phytagel | 3 | g |

Add distilled water to 900 ml, adjust pH to 5.9 with 1N potassium hydroxide, boil the solution, add water to a final volume 1000 ml and distribute the solution into 50 ml-triangular flasks (25 ml/flask) followed by sealing and sterilization.

2) Subculture Medium

| | | |
|---|---|---|
| N6max concentrated solution (10X) | 100 | ml |
| N6mix concentrated solution (100X) | 10 | ml |
| Fe²⁺EDTA stock solution (100X) | 10 | ml |
| Vitamin stock solution (100X) | 10 | ml |
| 2,4-D stock solution | 2.0 | ml |
| Proline | 0.5 | g |
| CH | 0.6 | g |
| Sucrose | 30 | g |
| Phytagel | 3 | g |

Add distilled water to 900 ml, adjust pH to 5.9 with 1 N potassium hydroxide, boil the solution, add water to a final volume 1000 ml and distribute the solution into 50 ml-triangular flasks (25 ml/flask) followed by sealing and sterilization.

3) Preculture Medium

| | | |
|---|---|---|
| N6max concentrated solution (10X) | 12.5 | ml |
| N6mix concentrated solution (100X) | 1.25 | ml |
| Fe²⁺EDTA stock solution (100X) | 2.5 | ml |
| Vitamin stock solution (100X) | 2.5 | ml |
| 2,4-D stock solution | 0.75 | ml |
| CH | 0.15 | g |
| Sucrose | 5 | g |
| Agarose | 1.75 | g |

Add distilled water to 250 ml, adjust pH to 5.6 with 1 N potassium hydroxide followed by seal and sterilization. Prior to use, heat and dissolve the medium, add 5 ml glucose stock solution and 250 μl AS stock solution and distribute the solution into dishes (25 ml/dish).

4) Coculture Medium

| | | |
|---|---|---|
| N6max concentrated solution (10X) | 12.5 | ml |
| N6mix concentrated solution (100X) | 1.25 | ml |
| Fe²⁺EDTA stock solution (100X) | 2.5 | ml |
| Vitamin stock solution (100X) | 2.5 | ml |
| 2,4-D stock solution | 0.75 | ml |
| CH | 0.2 | g |
| Sucrose | 5 | g |
| Agarose | 1.75 | g |

Add distilled water to 250 ml, adjust pH to 5.6 with 1 N potassium hydroxide followed by sealing and sterilization. Prior to use, heat and dissolve the medium, add 5 ml glucose stock solution and 250 μl AS stock solution and distribute the solution into dishes (25 ml/dish).

5) Suspension Culture Medium

| | | |
|---|---|---|
| N6max concentrated solution (10X) | 5 | ml |
| N6mix concentrated solution (100X) | 0.5 | ml |
| Fe²⁺EDTA stock solution (100X) | 0.5 | ml |
| Vitamin stock solution (100X) | 1 | ml |
| 2,4-D stock solution | 0.2 | ml |
| CH | 0.08 | g |
| Sucrose | 2 | g |

Add distilled water to 100 ml, adjust pH to 5.4 and distribute the solution into two 100 ml-triangular flasks followed by sealing and sterilization. Prior to use, add 1 ml glucose stock solution and 100 μl AS stock solution.

6) Selection culture medium

| | | |
|---|---|---|
| N6max concentrated solution (10X) | 25 | ml |
| N6mix concentrated solution (100X) | 2.5 | ml |
| Fe²⁺EDTA stock solution (100X) | 2.5 | ml |
| Vitamin stock solution (100X) | 2.5 | ml |
| 2,4-D stock solution | 0.625 | ml |
| CH | 0.15 | g |
| Sucrose | 7.5 | g |
| Agarose | 1.75 | g |

Add distilled water to 250 ml and adjust pH to 6.0 followed by sealing and sterilization. Prior to use, dissolve the medium, add 250 μl HN and 400 ppm CN and distribute the medium into dishes (25 ml/dish).

7) Predifferentiation Medium

| | | |
|---|---|---|
| N6max concentrated solution (10X) | 25 | ml |
| N6mix concentrated solution (100X) | 2.5 | ml |
| Fe²⁺EDTA stock solution (100X) | 2.5 | ml |
| Vitamin stock solution (100X) | 2.5 | ml |
| 6-BA stock solution | 0.5 | ml |
| KT stock solution | 0.5 | ml |
| NAA stock solution | 50 | μl |
| IAA stock solution | 50 | μl |
| CH | 0.15 | g |
| Sucrose | 7.5 | g |
| Agarose | 1.75 | g |

Add distilled water to 250 ml, adjust pH to 5.9 with 1N potassium hydroxide followed by sealing and sterilization. Prior to use, dissolve the medium, add 250 μl HN and 200 ppm CN and distribute the medium into dishes (25 ml/dish).

8) Differentiation Medium

| | | |
|---|---|---|
| N6max concentrated solution (10X) | 100 | ml |
| N6mix concentrated solution (100X) | 10 | ml |
| Fe²⁺EDTA stock solution (100X) | 10 | ml |
| Vitamin stock solution (100X) | 10 | ml |
| 6-BA stock solution | 2 | ml |
| KT stock solution | 2 | ml |
| NAA stock solution | 0.2 | ml |
| IAA stock solution | 0.2 | ml |
| CH | 1 | g |
| Sucrose | 30 | g |
| Phytagel | 3 | g |

Add distilled water to 900 ml, adjust pH to 6.0 with 1 N potassium hydroxide, boil the solution, add water to a final volume 1000 ml and distribute the solution into 50 ml-triangular flasks (50 ml/flask) followed by sealing and sterilization.

9) Rooting Medium

| | | |
|---|---|---|
| MSmax concentrated solution (10X) | 50 | ml |
| MSmix concentrated solution (100X) | 5 | ml |
| Fe²⁺EDTA stock solution (100X) | 5 | ml |
| Vitamin stock solution (100X) | 5 | ml |
| Sucrose | 30 | g |
| Phytagel | 3 | g |

Add distilled water to 900 ml, adjust pH to 5.8 with 1 N potassium hydroxide, boil the solution, add water to a final volume 1000 ml and distribute the medium into rooting tubes (25 ml/flask) followed by sealing and sterilization.

5. The Growth of the Transgenic OsPP18-Overexpression T1 Lines Under Osmosis Stress.

In the present invention, the applicants used some transgenic rice plants as samples and conducted real time fluorescent quantitative analysis to study the expression level of OsPP18 gene. The procedure of RNA extraction, reverse transcription, and real time fluorescent quantitative analysis are the same as those provided in Example 2 (FIG. 6. B shows the result of the expression level test). The results demonstrate the obtaining of transgenic plants with significantly enhanced expression of OsPP18 gene as compared to wild type control.

In the present example, the applicants conducted the osmosis stress experiment by employing two transgenic T1 lines (No: OsPP18-OX-4 and OsPP18-OX-7) that overexpress the OsPP18 gene (sequence is set forth in SEQ NO: 1). The particular procedure is as follows: the seeds of the transgenic OsPP18-overexpression lines (OsPP18-OX-4, OsPP18-OX-7) were deshelled (treated with 70% alcohol for 1 minute, then treated with 0.15% HgCl₂ for 10 minutes, and finally washed with sterilized water for several times). The seeds were then allowed to germinate on ½ MS medium containing 50 mg/L hygromycin. The seeds of Zhonghua 11 (ZH11) were planted one day later on ½ MS medium that does not contain hygromycin. After 2-3 days, the seeds that germinated well and grew similarly were selected and transferred into ½ MS medium with or without 150 mM mannitol. After 10 days, the plants were photographed, and the plant height was measured (See FIG. 6. C). Since the overexpression plants and the control grew differently on the mannitol-free medium (FIG. 6. C. top), relative plant height (the length of the plants growing on the mannitol medium (FIG. 6. C. bottom) divided by the length of the plants growing on the mannitol-free medium (FIG. 6. C. top)) was used to compare the lines. The relative plant height of the overexpression plants is significantly higher than that of the wild type control (FIG. 6. D). The experiment was conducted in triplet. The results were the same. Therefore, overexpression of OsPP18 gene indeed can increase the drought resistance of the transgenic plant.

1-10. (canceled)

11. A DNA construct comprising a heterologous promoter operably linked to a polynucleotide encoding a protein phosphatase 2C (PP2C) polypeptide comprising an amino acid sequence at least 90% identical to SEQ ID NO: 2.

12. The DNA construct of claim 11, wherein said polynucleotide comprises the nucleotide sequence as set forth in SEQ ID NO: 1 from nucleotide position 38 to nucleotide position 1084.

13. The DNA construct of claim 12, wherein said polynucleotide comprises the nucleotide sequence as set forth in SEQ ID NO: 1.

14. The DNA construct of claim 11, wherein said promoter is a drought-inducible promoter.

15. A transgenic plant or plant part comprising the DNA construct of claim 11, wherein said plant is drought tolerant and said plant part comprises seed or cell.

16. The transgenic plant or plant part of claim 15, wherein said plant is selected from the group consisting of a corn, soybean, cotton, canola, rice, barley, oats, wheat, turf grass, alfalfa, sugar beet, sunflower, quinoa and sugar cane plant.

17. A method of improving drought resistance of a plant, comprising providing a transgenic plant comprising the DNA construct of claim 11, wherein expression of said polynucleotide is induced under drought condition thereby improving the drought resistance of the transgenic plant.

18. The method of claim 17, wherein said providing step comprises transforming a plant with said DNA construct thereby obtaining said transgenic plant.

19. The method of claim 17, wherein said plant is selected from the group consisting of a corn, soybean, cotton, canola, rice, barley, oats, wheat, turf grass, alfalfa, sugar beet, sunflower, quinoa and sugar cane plant.

20. A DNA construct comprising a heterologous promoter operably linked to a polynucleotide encoding a PP2C polypeptide comprising an amino acid sequence at least 90% identical to SEQ ID NO: 2, wherein said promoter is an ubiquitin promoter.

21. The DNA construct of claim 20, wherein said promoter is a corn ubiquitin promoter.

22. The DNA construct of claim 20, wherein said polynucleotide comprises the nucleotide sequence as set forth in SEQ ID NO: 1 from nucleotide position 38 to nucleotide position 1084.

23. The DNA construct of claim 22, wherein said polynucleotide comprises the nucleotide sequence as set forth in SEQ ID NO: 1.

24. A transgenic plant or plant part comprising the DNA construct of claim 20, wherein said plant is drought tolerant and said plant part comprises seed or cell.

25. The transgenic plant or plant part of claim 24, wherein said plant is selected from the group consisting of a corn, soybean, cotton, canola, rice, barley, oats, wheat, turf grass, alfalfa, sugar beet, sunflower, quinoa and sugar cane plant.

26. A method of improving drought resistance of a plant, comprising providing a transgenic plant comprising the DNA construct of claim 20, wherein said polynucleotide is overexpressed compared to a wild type plant, said transgenic plant exhibiting improved drought resistance compared to the wild type plant.

27. The method of claim 26, wherein said providing step comprises transforming a plant with said DNA construct thereby obtaining said transgenic plant.

28. The method of claim 26, wherein said plant is selected from the group consisting of a corn, soybean, cotton, canola, rice, barley, oats, wheat, turf grass, alfalfa, sugar beet, sunflower, quinoa and sugar cane plant.

29. A method of sowing, planting, or growing a plant with improved drought resistance, said method comprising the step of sowing, planting, or growing a transgenic plant comprising the DNA construct of claim 11.

30. A method of sowing, planting, or growing a plant with improved drought resistance, said method comprising the step of sowing, planting, or growing a transgenic plant comprising the DNA construct of claim 20.


SEQUENCE LISTING (12 sequences)

SEQ ID NO: 1 - DNA, 1162 bp, Oryza sativa
  CDS (38)..(1084): DNA sequence encoding OsPP18
ctcctccatccattcccctcctcctcctcctcctcccatgcatgggaggccgtcgaggccattagcttcctccccgtcgtcgtcgtcgtcccgcgtcttctccttcttcttggcgccgcgggttttcttgttcttggtggtggtggtggtcgtcgtcttcttgccgggtcggtcgtcttgctggtggctggagggcacggaggagttggaggaggagatggggtttgccggggactgctcaccggtcagcggtggcgggctcagtgaaaacggcaagttcagttatgggtatgcaagtgctcctgggaaaagggcttcaatggaggatttctacgagacgagaatcgatggtgttgatggagagaccattggattatttggcgtattcgacggccatggcggagctcgagcggctgaatatgtcaagcagcaccttttcagcaatttaattaagcatccaaagttcatcagtgatatcaagtccgctatcgctgaaacgtacaaccatacagattcagaatttctgaaagccgaaagtagtcacactagggatgctggctcaactgcctcaacagctattcttgtaggcgatcgcttgctggttgctaatgttggagattctagagcagttgtttgtagaggcggggatgcaattgcagtttcaagggatcacaaacccgaccagtcagatgagagacaaagaattgaggacgctgggggcttcgtgatgtgggctgggacgtggcgtgttggtggtgttcttgctgtttctcgagcatttggtgataagctattgaagcagtatgtagttgctgatccagagatcaaggaggagattgtcgatagctcccttgagttcctcatccttgctagcgatggactttgggatgttgtaagtaacaaggaagctgttgacatggtgaggcctattcaggatcccgaacaggcagcgaagaggcttctccaggaggcgtaccaaaggggtagcgccgataacatcaccgttgttattgtccgctttttggagggaacaacgactggtggtggaccaagtagggaggccgccagcgaccaaaactcatagtttctcccaggcagcagcatggcttgttcctgtctgtcatatctgatgctcaaggtagacgattacagtggcggcacc

SEQ ID NO: 2 - protein, 348 aa, Oryza sativa
  amino acid sequence of OsPP18
MHGRPSRPLASSPSSSSSRVFSFFLAPRVFLFLVVVVVVVFLPGRSSCWWLEGTEELEEEMGFAGDCSPVSGGGLSENGKFSYGYASAPGKRASMEDFYETRIDGVDGETIGLFGVFDGHGGARAAEYVKQHLFSNLIKHPKFISDIKSAIAETYNHTDSEFLKAESSHTRDAGSTASTAILVGDRLLVANVGDSRAVVCRGGDAIAVSRDHKPDQSDERQRIEDAGGFVMWAGTWRVGGVLAVSRAFGDKLLKQYVVADPEIKEEIVDSSLEFLILASDGLWDVVSNKEAVDMVRPIQDPEQAAKRLLQEAYQRGSADNITVVIVRFLEGTTTGGGPSREAASDQNS

SEQ ID NO: 3 - protein, 348 aa, Oryza sativa
  amino acid sequence of OsPP18 homolog
MHGRPSRPLASSSSSSSSRVFSFFLAPRVFLFLVVVVVVVFLPGRSSCWWLEGTEELEEEMGFAGDCSPVSGGGLSENGKFSYGYASAPGKRASMEDFYETRIDGVDGETIGLFGVFDGHGGARAAEYVKQHLFSNLIKHPKFISDIKSAIAETYNHTDSEFLKAESSHTRDAGSTASTAILVGDRLLVANVGDSRAVVCRGGDAIAVSRDHKPDQSDERQRIEDAGGFVMWAGTWRVGGVLAVSRAFGDKLLKQYVVADPEIKEEIVDSSLEFLILASDGLWDVVSNKEAVDMVRPIQDPEQAAKRLLQEAYQRGSADNITVVIVRFLEGTTTGGGPSREAASDQNS

SEQ ID NO: 4 - protein, 368 aa, Oryza sativa
  amino acid sequence of OsPP18 homolog
MHGRPSRPLASSSSSSSSRVFSFFLAPRVFLFLVVVVVVVFLPGRSSCWWLEGTEELEEEMGFAGDCSPVSGGGLRGKIQEEEEEEEKDDKFFMARENGKFSYGYASAPGKRASMEDFYETRIDGVDGETIGLFGVFDGHGGARAAEYVKQHLFSNLIKHPKFISDIKSAIAETYNHTDSEFLKAESSHTRDAGSTASTAILVGDRLLVANVGDSRAVVCRGGDAIAVSRDHKPDQSDERQRIEDAGGFVMWAGTWRVGGVLAVSRAFGDKLLKQYVVADPEIKEEIVDSSLEFLILASDGLWDVVSNKEAVDMVRPIQDPEQAAKRLLQEAYQRGSADNITVVIVRFLEGTTTGGGPSREAASDQNS

SEQ ID NO: 5 - protein, 365 aa, Oryza sativa
  amino acid sequence of OsPP18 homolog
MHGRPSRPLASSSSSSSSRVFSFFLAPRVFLFLVVVVVVVFLPGRSSCWWLEGTEELEEEMGFAGDCSPVSGGGLRGKIQEEEEEEEEEKDDNENGKFSYGYASAPGKRASMEDFYETRIDGVDGETIGLFGVFDGHGGARAAEYVKQHLFSNLIKHPKFISDIKSAIAETYNHTDSEFLKAESSHTRDAGSTASTAILVGDRLLVANVGDSRAVVCRGGDAIAVSRDHKPDQSDERQRIEDAGGFVMWAGTWRVGGVLAVSRAFGDKLLKQYVVADPEIKEEIVDSSLEFLILASDGLWDVVSNKEAVDMVRPIQDPEQAAKRLLQEAYQRGSADNITVVIVRFLEGTTTGGGPSREAASDQNS

SEQ ID NO: 6 - DNA, 480 bp, Artificial
  sequence flanking the OsPP18 T-DNA mutant 2D-21407
gnnatnccccagttgtcatgtattagccacatagcaaaaaaaatagcaccgtggtagtaagaatggaactcacctggtacctggtacctcggatccgtgttttcgtgttagggggaagatccaagaggaggaggaggaggaggagaaggatgacaagtttttcatggcgcggtcaggggctttgtgcttgatttgattggcggctaactatcgagtagtgattcttgaatcagaggctaggacatgaggtttcagtggcggattttgaattagatctgcccgattttgctctgaattagcttagcctaatcgggttccagctcctggctggtgtgaggatgtggccatgattttgaatttgaggagtgtttgaaaacnaaaagtctaatcctttgtaagggcatatggtanaggaattaacctttttcttgtttgatgtgtctgtgtgnancanctgatttttatggantgttnttantt

SEQ ID NO: 7 - DNA, 20 bp, Artificial
  OsPP18-F primer
cacaaacccgaccagtcaga

SEQ ID NO: 8 - DNA, 17 bp, Artificial
  OsPP18-R primer
cgtcccagcccacatca

SEQ ID NO: 9 - DNA, 21 bp, Artificial
  AF primer for amplifying a 76 bp fragment of the rice Actin1 gene
tggcatctctcagcacattcc

SEQ ID NO: 10 - DNA, 20 bp, Artificial
  AR primer for amplifying a 76 bp fragment of the rice Actin1 gene
tgcacaatggatgggtcaga

SEQ ID NO: 11 - DNA, 26 bp, Artificial
  OSPP18FLF-sequence specific primer with additional KpnI cleavage site
atcggtaccctcctccatccattccc

SEQ ID NO: 12 - DNA, 25 bp, Artificial
  OSPP18FLR-sequence specific primer with additional BamHI cleavage site
atcggtaccggtgccgccactgtaa